(19) United States
(12) Patent Application Publication
(10) Pub. No.: US 2022/0095996 A1
(43) Pub. Date: Mar. 31, 2022

(54) WEARABLE MEDICAL DEVICE

(71) Applicant: Melissa Patterson, San Diego, CA (US)

(72) Inventor: Melissa Patterson, San Diego, CA (US)

(21) Appl. No.: 17/484,109

(22) Filed: Sep. 24, 2021

Related U.S. Application Data
(60) Provisional application No. 63/084,002, filed on Sep. 27, 2020.

(51) Int. Cl. A61B 5/00; A41C 3/00; A41D 13/12

(52) U.S. Cl. CPC A61B 5/4312; A61B 5/6804; A61B 5/0002; A61B 5/7267; A61B 2562/164; A41C 3/0064; A41D 13/1281; A61B 2562/04; A61B 5/4842

(57) ABSTRACT

A medical device for early detection of breast cancer is provided. Embodiments of the application incorporate a medical device (e.g., formed as a sports bra), one or more user devices, and an analytics computing device. The medical device is incorporated with a plurality of sensors to detect changes in density (or other metrics) of the breast tissue. The medical device is placed snuggly over the breast tissue to generate measurements by the plurality of sensors. The measurements are transmitted to the analytics computing device to analyze over a time period. When the measurements exceed a threshold value, the analytics computing device may perform an action, including transmitting an electronic communication to a physician user or a patient user (e.g., to identify a potential issue, to transfer the measurement data, to recommend an action to the patient user).

### CROSS-REFERENCE TO RELATED APPLICATIONS

This is a non-provisional patent application of U.S. Patent Application No. 63/084,002, filed Sep. 27, 2020, which is hereby incorporated by reference for all purposes.

### BACKGROUND

Breast cancer is pervasive. Early detection for breast cancer includes doing monthly breast self-exams and visiting a doctor to perform clinical breast exams and mammograms. However, monthly breast self-exams are performed by individuals that might not know what to look for or might not perform these exams regularly. Additionally, these users might not be able to visit the doctor physically due to the pandemic or other mobility concerns. Better early detection methods are needed.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 illustrates a breast cancer detection system, in accordance with some embodiments of the application.

FIGS. 2 and 3 illustrate examples of a front view of a medical device in a breast cancer detection system, in accordance with some embodiments of the application.

FIG. 4 illustrates an example of a back view of a medical device, in accordance with some embodiments of the application.

FIGS. 5 and 6 illustrate internal layers of a medical device, in accordance with some embodiments of the application.

FIGS. 7A-7D illustrate a medical device and breast tissue, in accordance with some embodiments of the application.

FIG. 8 illustrates an analytics computing device in a breast cancer detection system, in accordance with some embodiments of the application.

FIGS. 9-11 illustrate example data stores in communication with an analytics computing device, in accordance with some embodiments of the application.

FIG. 12 is an illustrative mapping of breast tissue by a breast cancer detection system, in accordance with some embodiments of the application.

FIGS. 13A-13C are illustrative electronic communications, in accordance with some embodiments of the application.

FIGS. 14-15 are illustrative processes performed by devices in the breast cancer detection system, in accordance with some embodiments of the application.

The figures are not intended to be exhaustive or to limit the invention to the precise form disclosed. It should be understood that the invention can be practiced with modification and alteration, and that the disclosed technology be limited only by the claims and the equivalents thereof.

### DETAILED DESCRIPTION OF THE DRAWINGS

Embodiments of the application provide a breast cancer detection system by incorporating a medical device (e.g., formed as a sports bra), one or more user devices, and an analytics computing device. The medical device is incorporated with a plurality of sensors to detect changes in density (or other metrics) of the breast tissue. The medical device is placed snuggly over the breast tissue to generate measurements by the plurality of sensors. The measurements are transmitted to the analytics computing device to analyze over a time period. When the measurements exceed a threshold value, the analytics computing device may perform an action, including transmitting an electronic communication to a physician user or a patient user (e.g., to identify a potential issue, to transfer the measurement data, to recommend an action to the patient user). In some examples, the analytics computing device may be incorporated with a physician's office to update a patient's medical records or to notify the physician of the measurements.

Although breast tissue and a medical device (shaped like a sports bra) are illustrated and described throughout the disclosure, various types of tissue, body parts, and medical devices may be implemented. For example, any location where a lump (e.g., formed under the skin and pushed outward so that it is measurable at the surface of the skin, etc.) or change in tissue (e.g., soft, squishy, flexible to stiff, coarse, inflexible, etc.) may benefit from the medical device described herein. In either of these instances, the medical device may apply sensors to encompass the area of the body that may grow the lump or change in tissue.

Technical embodiments are realized throughout the disclosure. For example, standard systems do not incorporate a medical device operated by the user with a breast cancer detection system. The user must visit a physician's office to perform a clinical exam or mammogram. Even if the user has time to perform self-examinations, they are not trained to identify what differences in tissue measurements mean from month to month (or other time periods). With more frequent use, more measurement readings are obtained by the medical device described herein, thus allowing for more accurate readings and improving the data analytics overall. These analytics and results created were previously unattainable. Users are empowered to use the medical device with everyday life, which can detect changes in breast tissue more frequently and with more precision.

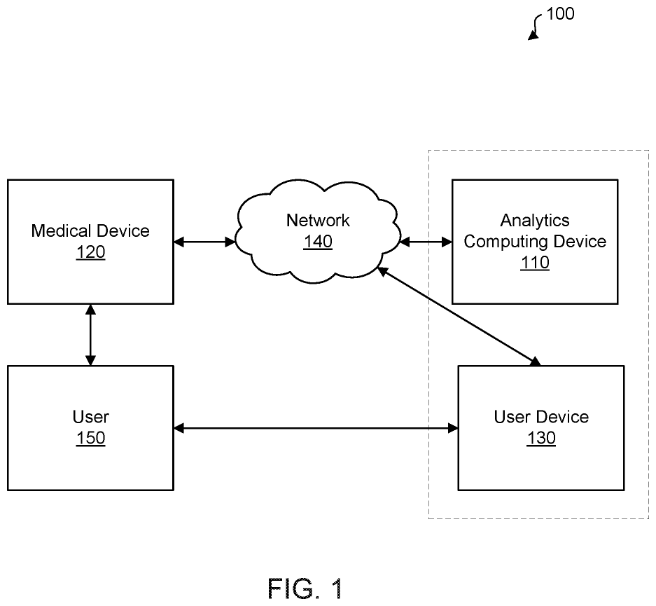

FIG. 1 illustrates a breast cancer detection system, in accordance with some embodiments of the application. In illustration 100, a breast cancer detection system is provided. The breast cancer detection system can include, for example, analytics computing device 110 in communication with medical device 120 and user device 130 via one or more networks 140. For example, analytics computing device 110 may communicate with medical device 120 via network 140 (e.g., Internet, closed network, short-range wireless interconnection, wired connection with user device 130, etc.) to transmit measurements generated by sensors of medical device 120.

In another example, medical device 120 may transmit measurements to user device 130 via a first network 140 (e.g., near field communication (NFC), Bluetooth®, or other wired/wireless communication) and user device 130 may transmit measurements to analytics computing device 110 via a second network 140 (e.g., Internet). User 150 may operate medical device 120 by turning on medical device 120 (e.g., activating a battery embedded in medical device 120) or by putting on medical device 120 (e.g., to apply pressure to the sensors and initiate the process of generating measurements).

In another example, analytics computing device 110 may be embedded as a software application or cloud-based service at user device 130, such that analytics computing device 110 and user device 130 are a single device (as illustrated by the dashed line in FIG. 1). User device 130 may communicate via a first network 140 (e.g., near field communication (NFC), Bluetooth®, or other wired/wireless communication) with medical device 120 and user device 130 may analyze measurements locally at user device 130 using components of analytics computing device 110 incorporated in the software application, as described throughout the disclosure.

User device 130 may comprise a mobile device operated by user 150, including a smartphone, laptop computer, desktop computer, and the like. User device 130 may include customary device components of a mobile device, including an antenna, camera, battery, graphical user interface, memory, computer readable media, processor, and the like. User device 130 is configured to receive electronic communications from analytics computing device 110. User device 130 is also configured to provide the electronic communications at a graphical user interface to display information (e.g., for user 150).

User 150 may operate user device 130 to receive measurements from medical device 120 (e.g., via antennas at each medical device 120 and user device 130), transmit measurements to analytics computing device 110, or receive electronic communications from analytics computing device 110 regarding the modeling or measurements. Any of these transmissions may be initiated automatically, as described herein.

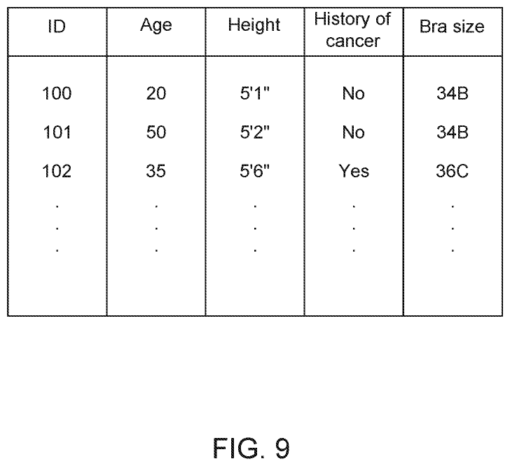

In some examples, user 150 may operate user device 130 to provide user information for a user profile and/or register to access analytics computing device 110 (either as an embedded software application at user device 130, as a standalone device accessible via network 140, or a cloud-implemented service, etc.). As part of the registration process, user may provide biographical and/or health information that may be stored in a user profile (discussed with FIG. 9). Analytics computing device 110 may store the user profile with a unique identifier of the user.

The user identifier may link to medical device 120 to user device 130 and user 150 as well. For example, when medical device 120 is powered on within a proximate distance to user device 130, the two devices may perform a handshake operation. Medical device 120 may transmit a beacon with identifying information (e.g., device identifier, number of sensors, location of sensor by sensor identifier, etc.) that is received by user device 130 (e.g., via a first network, NFC, Bluetooth®, etc.). User device 130 may receive the beacon and, in some examples, transmit a response to medical device 120. User device 130 may parse the beacon to determine the identifying information of medical device 120, and may transmit the identifying information to analytics computing device 110. Analytics computing device 110 may add the identifying information of medical device 120 to the user profile associated with user device 130 to correlate medical device 120, user device 130, and user 150 with the user profile.

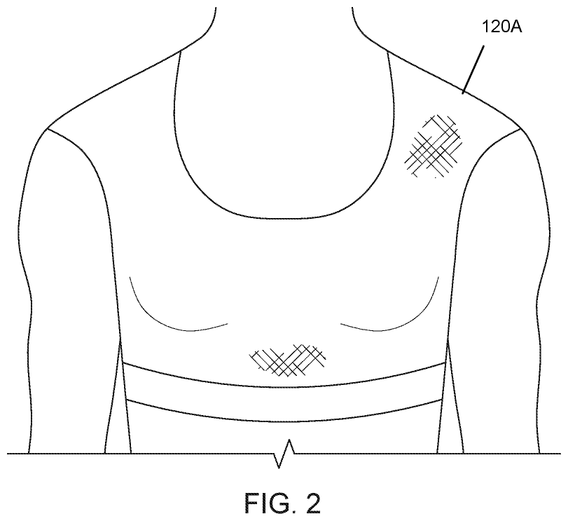

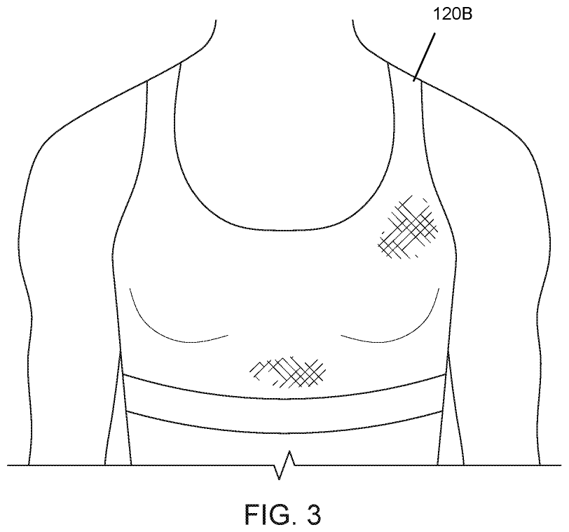

FIGS. 2 and 3 illustrate examples of a front view of a medical device in a breast cancer detection system, in accordance with some embodiments of the application. Medical device 120 (illustrated as first embodiment medical device 120A and second embodiment medical device 120B) may be formed as a bra that covers the supportive tissue (dense breast tissue) and the fatty tissue (non-dense breast tissue) of the breast area.

In FIG. 2, medical device 120A may cover the breast tissue on the front of the body, side, and back. Additional coverage over the shoulder may ensure that the fabric of medical device 120A covers the armpit or axilla area. In FIG. 3, medical device 120B may not include the additional fabric, but may still cover the armpit or axilla area. In either embodiment, medical device 120 may cover the breast tissue where breast cancer can traditionally form. This may include as much breast tissue as possible for more accurate data measurements.

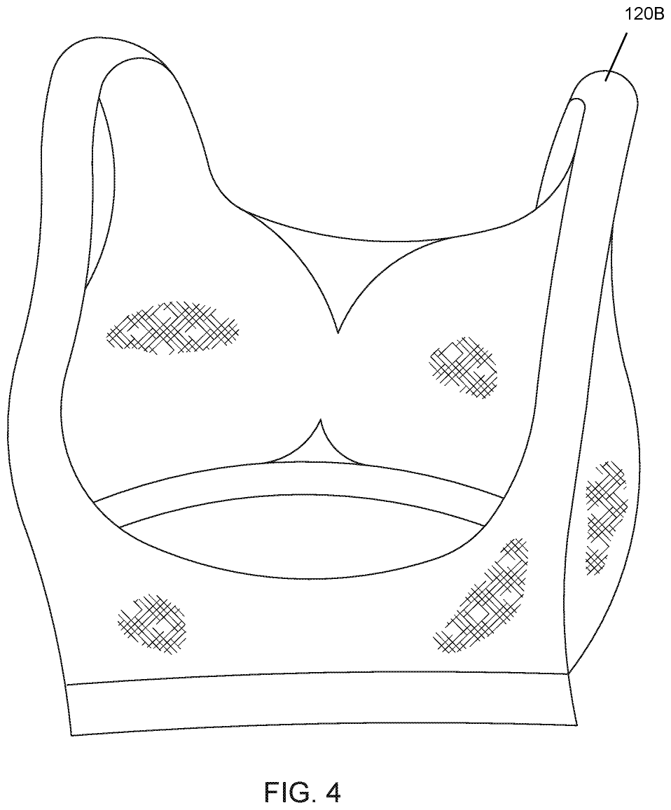

FIG. 4 illustrates an example of a back view of a medical device, in accordance with some embodiments of the application. The interior view of medical device 120B may show that additional fabric is used to cover additional areas of the breast tissue that standard bras may not cover. This may include the space between the breasts where breast cancer may traditionally form.

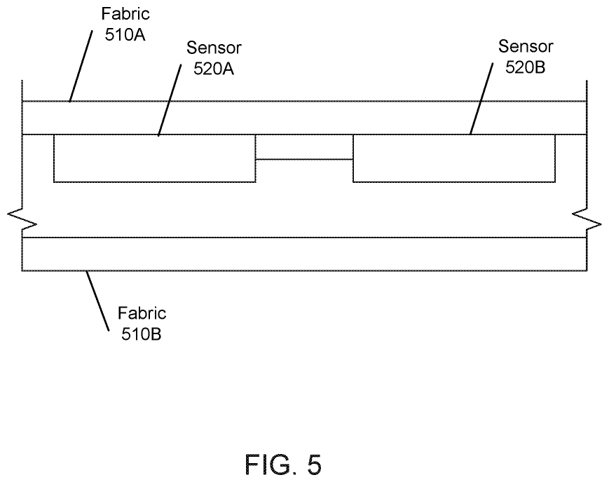

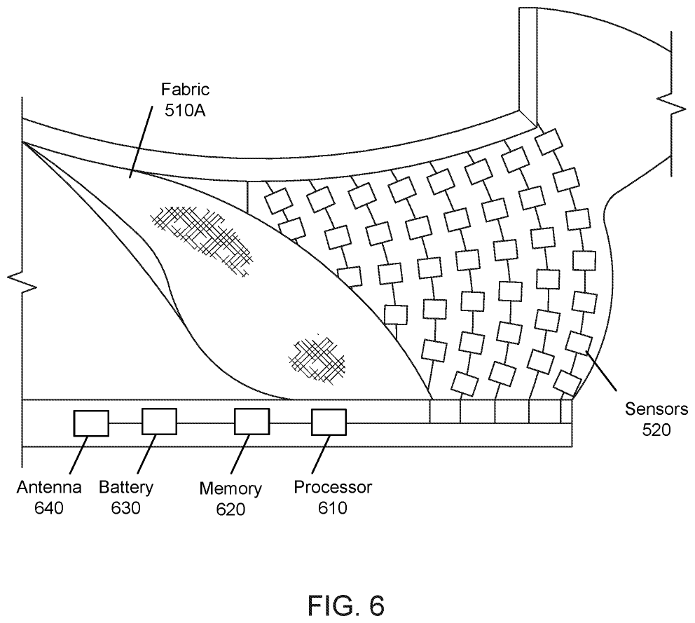

In each of these examples, fabric is provided to cover various sensors embedded within medical device 120. Fabric may be sewn to the structure of medical device 120 so that a first surface of the fabric communicatively connects with the skin of the user and an opposite surface of the fabric communicatively connects with one surface of the sensors. Various types of fabric may be used, including cotton, jersey, silk, satin, denim, velvet, thin and flexible polymer, or other fabrics that may help cover the sensors and the skin. Additional detail is provided with FIGS. 5 and 6.

FIGS. 5 and 6 illustrate internal layers of a medical device, in accordance with some embodiments of the application. In FIG. 5, two fabric layers 510 of medical device 120 are illustrated with a layer of sensors 520 between the fabric layers 510. The plurality of sensors 520 can be formed in a lattice or mesh within the fabric layers 510 of the medical device, as illustrated in FIG. 6.

Sensors 520 may comprise gauge-based pressure sensors, pressure transducer, pressure transmitter, pressure sender, pressure indicator, piezometer, manometer, or other similar sensor. Each sensor 520 may generate a measurement of pressure for an area surrounding the sensor, which may be based on the pressure produced between placing medical device 120 on the body of user 150 and measuring the resistance provided by the breast tissue.

In some examples, sensors 520 may measure density in the breast tissue. Each sensor may generate a measurement of density for an area around the sensor location of user 150 based on the density measurement produced by placing medical device 120 on the body and the detection of the density in the breast tissue generated by the sensor 520.

Sensors 520 may be adhered to fabric 510 to form a lattice or mesh of sensors to form the outline of medical device 120. In other examples, sensors 520 may be adhered to other sensors (e.g., first sensor 520A adhered to fabric 510A, second sensor 520B adhered to fabric 510A, etc.). Any adhesive is permissible. Fabric 510 with the lattice or mesh of sensors may form the outline of medical device 120.

The lattice or mesh of sensors 520 may be communicatively connected to each other, forming a plurality of connected sensors. In some examples, the angles between line segments connecting nearest neighbor points may approximately equal right angles, and the lengths of these line segments between nearest neighbor points may approximately be equal. As illustrated in FIG. 6, some line segments between sensors are slightly off of right angles in order to more closely form to the shape of the user's body.

In FIG. 5, two fabric layers 510 of medical device 120 are illustrated with a layer of sensors 520 between the fabric layers 510. Two fabric layers 510 of medical device 120 may differ. First fabric layer 510A may communicatively connect with the skin of the user and an opposite surface of first fabric layer 510A communicatively connects with one surface of the sensors 520. First fabric layer 510A that communicatively connects with the skin may be thin to allow pressure measurements to be sensed by the one or more sensors through the fabric. In some embodiments, this first fabric layer 510A is removed completely to allow for more accurate measurements. Second fabric layer 510B may communicatively connect with a second surface of sensors 520 and an opposite surface of second fabric layer 510B communicatively connects with the outer environment (e.g., the inside of the user's shirt, etc.). In some embodiments, this second fabric layer 510B is removed completely to allow for easier access to sensors 520.

In embodiments where first fabric layer 510A and/or second fabric layer 510B are implemented with medical device 120, either fabric layer 510A may be substantially tight to provide resistance against sensors 520 in the instance that a measurement value is received from the breast tissue. For example, the breast tissue may change over a time period to increase the density at a first location of a sensor from the plurality of sensors 520. Second fabric layer 520B may provide resistance so that, when the sensor physically pushes back in response to the increased pressure from the breast tissue and toward second fabric layer 520B, the fabric will provide resistance. The sensor may more accurately measure the pressure received from the breast tissue corresponding with the physical location of the sensor based on the resistance provided by the fabric.

In embodiments where first fabric layer 510A and/or second fabric layer 510B are removed, sensors 520 may form a lattice or mesh with each other, and while user 150 is wearing medical device 120, sensors 520 may be placed against the user's skin. Sensors 520 may be adhered to each other (e.g., directly adhered, adhered via conductive wires between the sensors, etc.) and the edges of the lattice or mesh of sensors 520 may form the outline of medical device 120. When pressure is applied to sensors from the user's skin, the resistance may be provided by surrounding sensors. Each of the sensor that is closest to the physical location of the breast tissue that provides the pressure data may also receive pressure data based on the lattice or mesh configuration of the sensors. In this case, the sensor and surrounding sensors may all measure the increased pressure received from the breast tissue corresponding with the physical location of the sensor. This may identify a wider area for a doctor checkup, but may still identify increased pressure over a time period at the particular location.

Sensor measurements may be transmitted along the line segments of the lattice or mesh of sensors 520 to processor 610, as illustrated in FIG. 6. Sensor measurements may be received by processor 610 and stored (either temporarily or permanently) in memory 620. Processor 610 may comprise a microprocessor, controller, or other control logic, which is connected to a bus, although any communication medium can be used to facilitate interaction with other components of medical device 120 or communicate externally (e.g., user device 130, etc.) via antenna 640.

Memory 620 may comprise random-access memory (RAM) or other dynamic memory to store information and instructions to be executed by processor 610. Memory 620 may be configured to store temporary variables or other intermediate information during execution of instructions to be executed by processor 610. Memory 620 may be connected to a bus for storing static information and instructions. Processor 610 may execute the computer-implemented instructions to receive the sensor measurements from sensors 520 and transmit them via antenna 640 to a second device (e.g., user device 130, etc.).

The sensor measurements may be transmitted in accordance with rules executed by processor 610. For example, the sensor measurements may be received as pressure is applied to a threshold number of sensors in the layer of sensors 520 (e.g., a baseline measurement, at least 70% of the sensors identifying some pressure which shows that the user is wearing medical device 120, etc.). Once the threshold number of sensors in the layer of sensors 520 detects a pressure measurement, processor 610 may determine the measurement (e.g., after a predetermined time period, like ten seconds, etc.) corresponding with each sensor and transmit the measurements and unique sensor identifier to user device 130 or analytics computing device 110 (or first to user device 130 via near field communication (NFC), and then transmit to analytics computing device 110 via network 140, etc.).

Battery 630 may comprise a standard battery or wearable battery, either of which may provide power to processor 610, memory 620, sensors 520, and antenna 640, or to charge one or more capacitors incorporated with medical device 120. In some examples, battery 630 may be charged via a power cable being plugged into the wall (while medical device 120 is not in use) and a converter, if needed. In some examples, graphene (e.g., two-dimensional carbon) and other related materials can be directly incorporated into medical device 120 to produce the charge.

Antenna 640 is also embedded with medical device 120. Antenna 640 may comprise a radio frequency (RF) front end design tuned for multiband or single band applications with single or multiple feeds. This may include a dual band GPS/Bluetooth® antenna (1 feed or 2 feeds), multiband 4G antenna using 1 feed or 2 feeds (1 for low band, 1 for high band), or 5G antenna. Antenna 640 may be channeled through the Industrial/Scientific/Medical (ISM) band. Antenna 640 may be communicatively coupled with user device 130 via a wireless network to transmit electronic communications between the two devices.

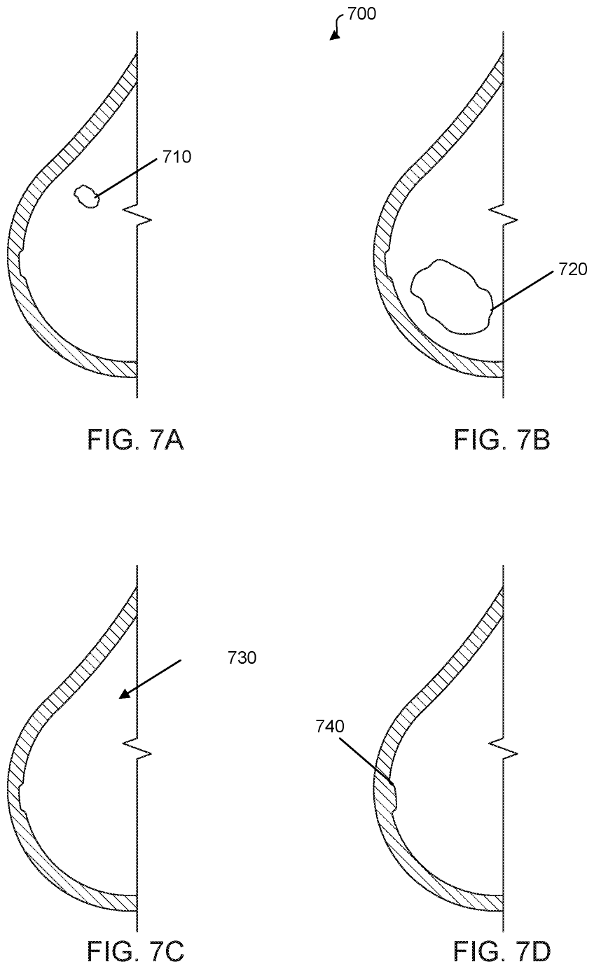

FIGS. 7A-7D illustrate a medical device and breast tissue, in accordance with some embodiments of the application. Standard breast tissue may comprise fatty, non-dense breast tissue. In some examples, breast tissue may comprise scattered areas of fibro-glandular density with some scattered areas of density. In other examples, breast tissue may comprise heterogeneously dense tissue with some areas of non-dense tissue. In still other examples, the breast tissue may be extremely dense.

In any of these instances, medical device 120 may generate measurements of the breast tissue to form a baseline model of the breast tissue using the lattice or mesh of sensors 520 that cover the breast tissue. Sensors 520 may continue to generate measurements over time. The additional measurements may identify changes to the baseline model of breast tissue, creating a unique mapping of how the breast tissue (at the particular location) changes during various time periods (e.g., daily, monthly, etc.). Some of these changes over time may correspond with a monthly cycle of the user and are expected changes in the breast tissue. Some changes may be indications of breast cancer, as illustrated in FIGS. 7A-7D.

In FIG. 7A, a hard or soft lump 710 has formed in the illustrative breast tissue 700. Medical device 120 may measure the slowly progressing density changes between the fatty breast tissue and lump 710 formed within the breast tissue over a time period. When the breast tissue is more dense, the changes in density may be measured by the sensors at a lesser degree of change than breast tissue that is mostly fatty.

In FIG. 7B, thickened skin 720 has formed with the breast tissue 700. Like in FIG. 7A, medical device 120 may measure the slowly thickening of the skin that covers the breast tissue over time.

In FIG. 7C, the shape or size of the breast 730 changes over a time period. These changes may include bulges, dimples, flatting or shrinking of the skin, swelling, or other changes to the breast tissue and/or surrounding skin. Sensors 520 incorporated with medical device 120 may measure these changes, which often slowly occur over a time period.

In FIG. 7D, the nipple 740 has inverted or otherwise changed in the illustrative breast tissue 700. Medical device 120 may measure outward protruding to inward protruding by the sensors that cover the nipple area.

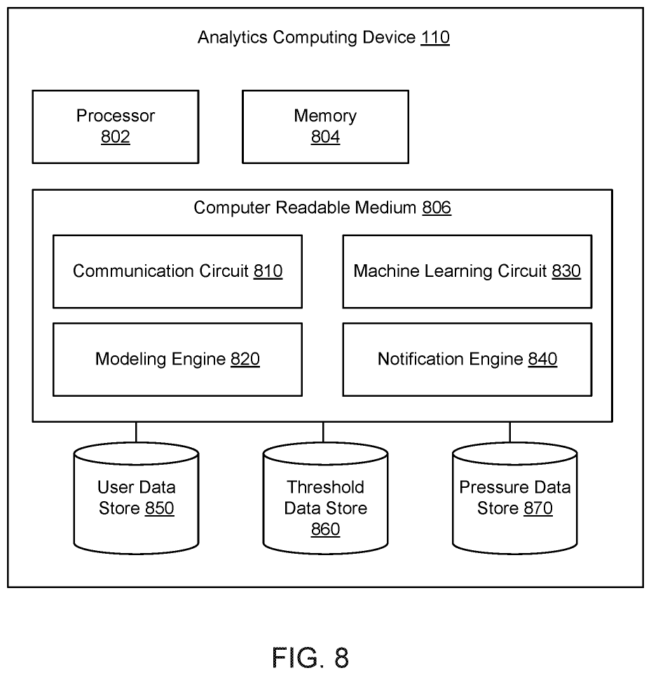

FIG. 8 illustrates an analytics computing device in a breast cancer detection system, in accordance with some embodiments of the application. Analytics computing device may include processor 802, memory 804, and computer readable media 806. Processor 802 may be configured to execute machine-readable instructions stored in memory to perform various operations described herein.

Communication circuit 810 is configured to receive electronic communications from medical device 120 and/or user device 130. The communications may be transmitted by an antenna embedded in either device.

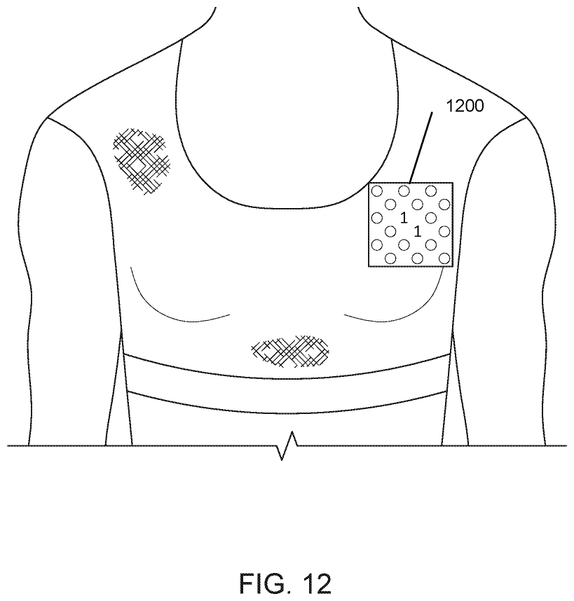

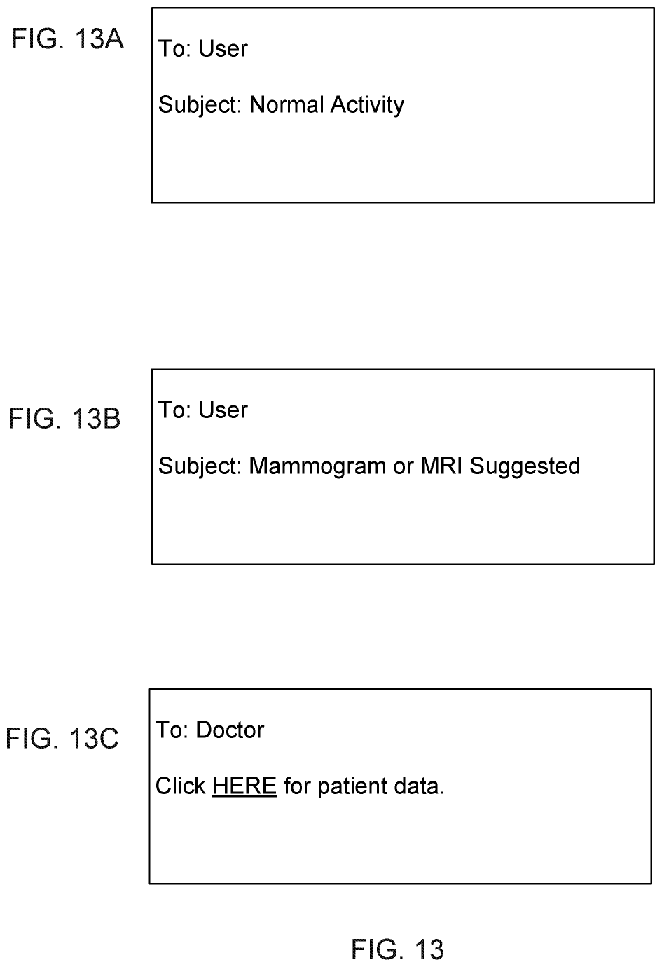

Communication circuit 810 is also configured to transmit electronic communications to user device 130. User device 130 may be operated by a patient user or physician user. In either example, the electronic communication may comprise a notification to seek additional medical care, capture an image of the breast tissue (e.g. using a camera embedded with user device 130, etc.), generate a model of the breast tissue (e.g., using medical device 120, as illustrated in FIG. 12), measure the breast tissue (e.g., providing shirt size, bra size, or other information in association with a user profile, etc.), and the like. Illustrative examples of these electronic communications are provided with FIGS. 13A-13C.

Modeling engine 820 is configured to generate a model of the breast tissue using a layout of sensors 520 incorporated with medical device 120. An illustrative model is provided with FIG. 12. As discussed herein, sensors 520 may be formed as a lattice or mesh with a proximate distance between each sensor, as illustrated with FIG. 6.

Each sensor may correspond with an expected area of the breast tissue, for example, based on the shape and layout of sensors incorporated with medical device 120. As an illustrative example, sensors may be adhered or sown into medical device 120 at predetermined locations. These locations may include, for example, a first plurality of sensors mapped to the bottom of the medical device near an elastic band that fits around the user's upper waist to measure tissue changes in that area of the user, and a second plurality of sensors around the arm holes of the medical device to measure tissue changes around the user's armpit area. In some examples, sensors may be located to correspond with customarily fatty tissue of the user's breasts where changes in breast tissue traditionally occur, based on the layout of medical device 120 for fitting around breast tissue.

Machine learning circuit 830 is configured to receive inputs to a trained machine learning (ML) model and produce outputs that associate the inputs with a classification category and score. The inputs may correspond with the measurements generated by the sensors in medical device 120. The trained machine learning model may comprise weights and biases that align the inputs with one or more classification categories in a supervised machine learning model. The output of the ML model may associate the input with one or more classification categories. The classification categories may correspond with different types of breast cancer (e.g., potential issue, levels early/late, sizes of dense tissue large/small, etc.), normal changes in the breast tissue during a monthly cycle of the user, or other categories. The output may also comprise a score associating the sensor measurements with the score of the likelihood that the inputs correlate with each classification category.

The training of the ML model may include measurements generated by sensors of breast tissue where breast cancer is present and not present. The training may teach the ML model the rate of progression of the breast cancer and how the breast tissue changes over a time period, when the resulting state of the breast tissue includes the breast cancer or does not include the breast cancer.

Machine learning model 830 (with modeling engine 820) may generate output corresponding with each sensor of medical device 120. The output may identify a one or zero, for example, at the particular location of the sensor, although other output examples are available without diverting from the essence of the disclosure.

The “1” output may identify a likelihood over a threshold value that the particular area corresponding with the sensor comprises a hard or soft lump and the “0” output may identify a likelihood less than a threshold value that the particular area corresponding with the sensor comprises a hard or soft lump. The output may be generated for each sensor location as illustrated in FIGS. 7A-7D. For example, the input (e.g., sensor measurements, location, growth or increased density over a time period, etc.) may be provided to multiple trained ML models and the output may be generated for each model (e.g., classification category, etc.).

Notification engine 840 is configured to generate an electronic communication that includes information associated with the output of the ML model(s). The electronic communication may comprise, for example, the mapping of the one or zero to sensors 520 of medical device 120. In another example, the electronic communication may comprise potential areas of concern corresponding with a particular classification category and score. In yet another example, when the output includes a potential area of concern corresponding with a particular classification category and score, the electronic communication may aggregate any “1” results as an overall high likelihood result (e.g., greater than three or other threshold value, etc.). The aggregated result may correspond with an electronic communication to notify the user to visit a physician for additional clinical analysis (e.g., clinical exam, x-ray, magnetic resonance imaging (MM), surgical removal of the area and biopsy, etc.). The notification may also be transmitted directly to a user device of the physician to identify the patient's data (as permissible by law).

Analytics computing device 110 may also store various data, as illustrated with FIGS. 9-11. For example, data may be transmitted from medical device 120 (or user device 130 and received at analytics computing device 110 (by a wireless connection/antenna via network 140 or by a wired connection).

In FIG. 9, user data may be stored in user data store 850. User data may comprise, for example, a unique identifier corresponding with the user, age, height, history of cancer with the user (or familial connections with cancer), and/or bra size (e.g., corresponding with the size of medical device 120, and/or placement or number of sensors to detect breast cancer).

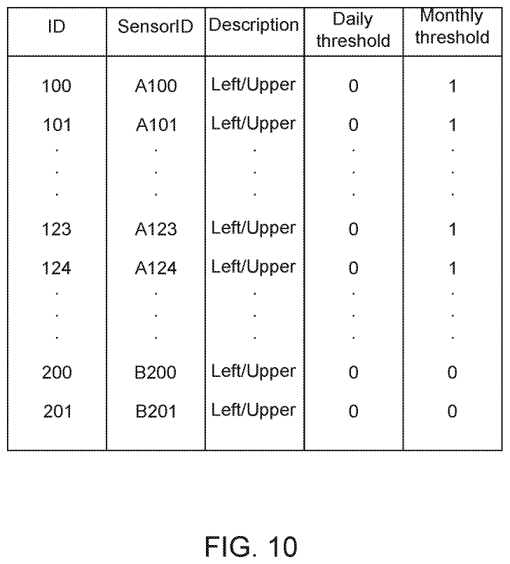

In FIG. 10, threshold data may be stored in threshold data store 860. Threshold data may comprise, for example, a unique identifier corresponding with a threshold value for a sensor (e.g., sensors may correspond with more than one threshold value), a unique identifier of each sensor in medical device 120, a written description of the location of the sensor within the lattice or mesh layout of the sensors in medical device 120, a daily threshold value, and/or a monthly threshold value. The written description of the location of the sensor 520 may be used to populate the electronic communication (e.g., notification) to the patient or physician user identifying the area of concern for breast cancer. The daily threshold value may indicate an acceptable change in measurement from day to day and the monthly threshold value may indicate an acceptable change in measurement from month to month. These values may be based on the location of the sensor 520 within medical device 120 and monthly hormonal or other expected changes with breast tissue.

In some examples, the threshold values may be adjusted based on the user data in user data store 850. For example, if the user has a history of cancer, the threshold values in threshold data store 860 may be adjusted to increase the sensitivity in identifying changes in the breast tissue. In another example, if the user is less than a certain age (e.g., 40, etc.) with no history of cancer, the threshold values in threshold data store 860 may be adjusted to decrease the sensitivity in identifying changes in the breast tissue.

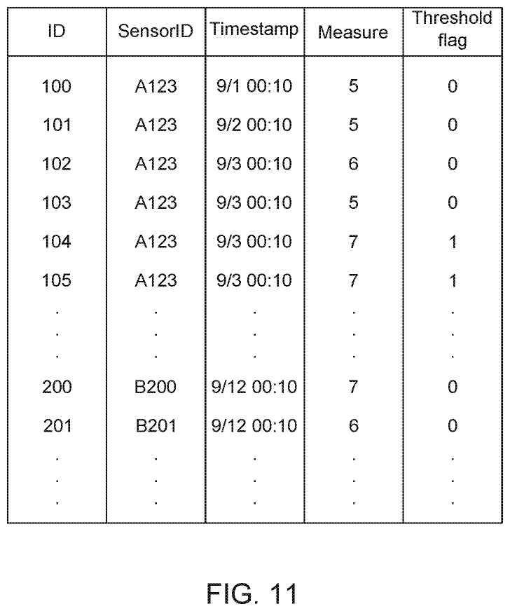

In FIG. 11, pressure data may be stored in pressure data store 870. The pressure data may comprise, for example a unique identifier of the pressure data for the sensor, a unique identifier of each sensor in medical device 120, timestamp that the measurement was generated by the sensor, measurement value, and threshold flag (e.g., whether the measurement value exceeded a threshold value).

Analytics computing device 110 may analyze data in the data stores to generate analytics in accordance with one or more rules. The rules may be received from a medical doctor or administrative user to help correlate the sensor values with issues that may indicate a potential for breast cancer. For example, when a measurement value from pressure data store 870 exceeds a monthly threshold value, the threshold flag may be activated (e.g., “1”). In another example, when a measurement value from pressure data store 870 fails to exceed a daily threshold value, the threshold flag may be deactivated (e.g., “0”).

FIG. 12 is an illustrative mapping of breast tissue by a breast cancer detection system, in accordance with some embodiments of the application. For example, modeling engine 820 may determine the threshold flags that have been activated and generate a map 1200 of the breast tissue to correspond with the physical location of the sensor in medical device 120. The activated threshold flags at the locations of the breast may correspond with whether the measurement generated by the sensor(s) at the location exceeded the threshold value.

In some examples, map 1200 may be automatically generated when the measurement exceeds the threshold value for a time period. For example, the measurement may exceed the threshold value for five days and not exceed the threshold on the sixth day. This may result in a mapping that does not overall exceed a threshold value and the mapping may identify a “0” indication. When the measurement exceeds the timing threshold value (e.g., twenty days, etc.), the mapping may indicate that the threshold value exceeds the measurement threshold value on the twenty-first day. These values may be altered by the physician user or patient user or in other embodiments automatically, without diverting from the scope of the disclosure.

FIGS. 13A-13C illustrate example electronic communications transmitted throughout the system, in accordance with some embodiments of the application. These electronic communications are illustrative and other electronic communications may indicate additional information stored in data stores 850, 860, 870, including, for example, the breast tissue location of the user that may or may not exceed the threshold value(s).

In FIG. 13A, the electronic communication may be generated when one or more measurement values do not exceed one or more threshold values. The electronic communication may identify “normal activity” in association with the threshold values.

In FIG. 13B, the electronic communication may be generated when one or more measurement values do exceed one or more threshold values. The measurement may exceed the threshold value for a given time period. The electronic communication may identify additional action that the user may perform, including requesting a mammogram, MM, or other medical procedure at a doctor's office.

In FIG. 13C, the electronic communication may be generated when one or more measurement values do or do not exceed one or more threshold values. The electronic communication may be transmitted to a doctor of a user and provide the medical data generated by medical device 120 to the doctor.

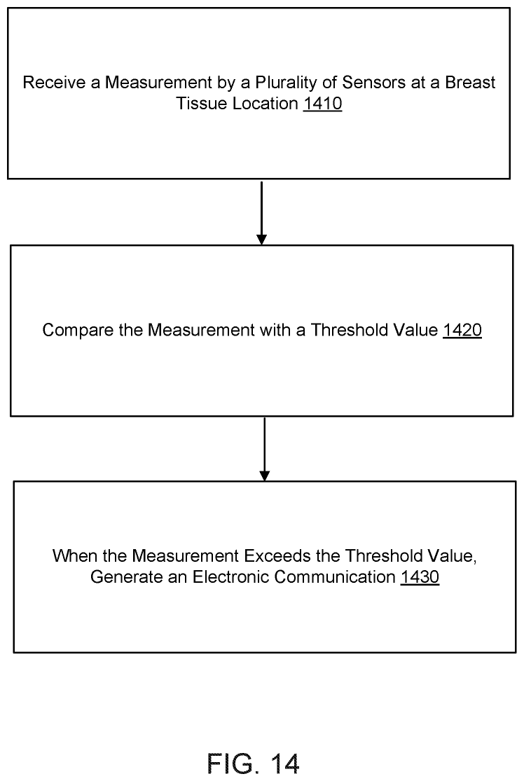

FIG. 14 illustrates a process for indicating changes in breast tissue over a time period, in accordance with some embodiments of the application. In some examples, analytics computing device 110 illustrated in FIG. 1 and FIG. 8 or user device 130 illustrated in FIG. 1 may perform the operations described herein.

At block 1410, receive a measurement by a plurality of sensors at a breast tissue location. For example, analytics computing device 110 (or user device 130 when it is integrated with a software application providing components of analytics computing device 110, etc.) may receive a measurement by at least one of the plurality of sensors at a breast tissue location of a user. The measurement may be received from medical device 120.

Medical device 120 may comprise plurality of sensors and a processor. The plurality of sensors may be formed as a lattice or mesh to communicate sensor measurements to the processor of the medical device. Medical device 120 may or may not include fabric layers, as discussed herein.

At block 1420, compare the measurement with a threshold value. For example, analytics computing device 110 (or user device 130) may compare the measurement with a threshold value. The threshold value may correspond with increased pressure at the particular area over a time period. As an illustrative example, a sensor measurement for a first sensor during a first time period may be of 120 mmHg and the same sensor providing a second sensor measurement for a second time period may be 130 mmHg. The difference between these two measurements is 10 mmHg, which may be compared with the threshold value of 5 mmHg. The measurement value (or difference in measurements over a time period) may exceed the threshold value.

At block 1430, when the measurement exceeds the threshold value, generate an electronic communication. For example, analytics computing device 110 (or user device 130) may generate the electronic communication associated with the comparison. The electronic communication may identify “normal activity” or a suggestion to visit a doctor's office for additional consideration from a medical professional.

In some examples, a map is generated corresponding with the comparison and location of the sensors, as illustrated in FIG. 12. The “1” output may identify a likelihood over a threshold value that the particular area corresponding with the sensor comprises a hard or soft lump and the “0” output may identify a likelihood less than a threshold value that the particular area corresponding with the sensor comprises a hard or soft lump

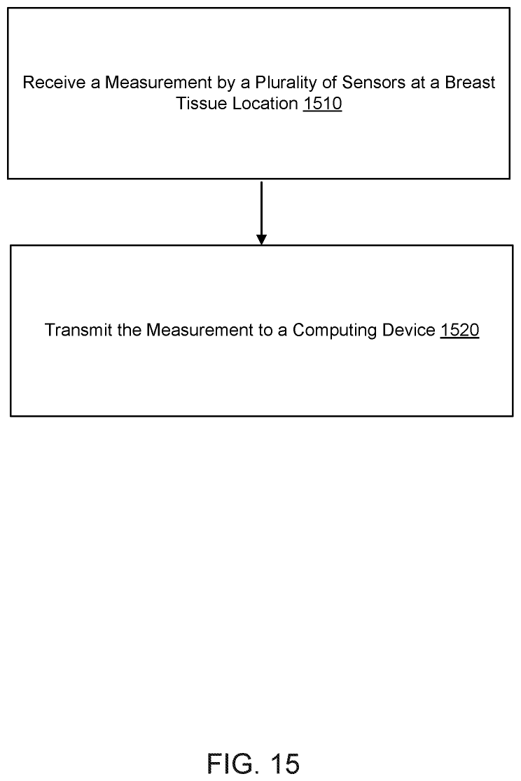

FIG. 15 illustrates a process for indicating changes in breast tissue over a time period, in accordance with some embodiments of the application. In some examples, medical device 120 illustrated in FIGS. 1-6 may perform the operations described herein.

At block 1510, a measurement may be received by at least one of the plurality of sensors. The sensor may correspond with a breast tissue location of a user. For example, one or more sensors incorporated with medical device 120 may generate a sensor measurement and provide the sensor measurement along line segments connecting the sensors. The processor of medical device may receive the sensor measurement for processing, storage, and/or transmission.

At block 1520, the measurement may be transmitted to a computing device. For example, the processor of medical device 120 may receive the sensor measurements and generate an electronic communication that includes the sensor measurements. After a handshake procedure, medical device 120 may transmit the electronic communication to user device 130 or analytics computing device 110 for further analysis.

The further analysis may include, for example, comparing the measurement with a threshold value, and when the measurement exceeds the threshold value for time period, generate an electronic communication associated with the comparison.

In the foregoing description, numerous details are set forth to provide an understanding of the present invention. However, it will be understood by those skilled in the art that the present invention may be practiced without these details. While the invention has been disclosed with respect to a limited number of embodiments, those skilled in the art will appreciate numerous modifications and variations therefrom. It is intended that the appended claims cover such modifications and variations as fall within the true spirit and scope of the invention.

1. A medical device comprising:
a fabric;
a plurality of sensors communicatively coupled with the fabric; and
a processor, wherein the processor is configured to execute machine readable instructions to:
receive a measurement by at least one of the plurality of sensors at a breast tissue location of a user; and
transmit the measurement to a computing device configured to:
compare the measurement with a threshold value; and
when the measurement exceeds the threshold value for time period, generate an electronic communication associated with the comparison.

2. The medical device of claim 1, wherein the medical device is in the form of a bra.

3. The medical device of claim 1, wherein the measurement is used to form a baseline model of the breast tissue location of the user.

4. The medical device of claim 1, wherein the measurement is used to form a unique mapping of how the breast tissue changes during various time periods.

5. The medical device of claim 1, wherein the plurality of sensors form a lattice or mesh of sensors that are communicatively coupled with the fabric.

6. The medical device of claim 1, wherein the plurality of sensors are adhered to the fabric.

7. The medical device of claim 1, wherein the plurality of sensors are sown to the fabric.

8. The medical device of claim 1, further comprising:
a battery configured to provide power to the plurality of sensors communicatively coupled with the fabric and the processor.

9. The medical device of claim 1, further comprising:
an antenna configured to wirelessly transmit the measurement to the computing device.

10. A computing device comprising:
a memory; and
one or more processors, wherein the processors are configured to execute machine readable instructions to:
receive a measurement by at least one of the plurality of sensors at a breast tissue location of a user from a medical device, wherein the medical device comprises: a plurality of sensors and a processor;
compare the measurement with a threshold value; and
when the measurement exceeds the threshold value for time period, generate an electronic communication associated with the comparison.

11. The computing device of claim 10, wherein the medical device is in the form of a bra.

12. The computing device of claim 10, the processors further configured to:
adjust the threshold value based on user data associated with the user.

13. The computing device of claim 10, the processors further configured to:
upon comparing the measurement with the threshold value, determine one or more threshold flags that have been activated; and
generate a map of the breast tissue in accordance with the one or more threshold flags that have been activated.

14. The computing device of claim 10, the processors further configured to:
provide the measurement as an input to a trained machine learning (ML) model, wherein weights and biases align the input with one or more classification categories; and
receive output from the trained ML model that associate the input with the one or more classification categories.

15. The computing device of claim 14, wherein the one or more classification categories are different types of breast cancer.

16. The computing device of claim 14, wherein the trained ML model is a supervised machine learning model.

17. The computing device of claim 14, wherein training the trained ML model teaches the rate of progression of breast cancer and/or how the breast tissue location changes over time, when the resulting state of the breast tissue location includes the breast cancer or does not include the breast cancer.

18. A computer-implemented method comprising:
receiving, by an analytics computing device, a measurement by at least one of the plurality of sensors at a breast tissue location of a user from a medical device, wherein the medical device comprises: a plurality of sensors and a processor;
comparing, by the analytics computing device, the measurement with a threshold value; and
when the measurement exceeds the threshold value for time period, generating, by the analytics computing device, an electronic communication associated with the comparison.

19. The computer-implemented method of claim 18, further comprising:
providing the measurement as an input to a trained machine learning (ML) model, wherein weights and biases align the input with one or more classification categories; and
receiving output from the trained ML model that associate the input with the one or more classification categories as different types of breast cancer.

20. The computer-implemented method of claim 19, wherein training the trained ML model teaches the rate of progression of breast cancer and/or how the breast tissue location changes over time, when the resulting state of the breast tissue location includes the breast cancer or does not include the breast cancer.
